(19) United States
(12) Patent Application Publication
(10) Pub. No.: US 2009/0048524 A1
(43) Pub. Date: Feb. 19, 2009

(54) IMPLANTABLE PRESSURE MEASURING UNIT AND CONFIGURATION FOR INTERNAL PRESSURE MEASUREMENT IN A BLOOD VESSEL

(76) Inventors: Hans-Jurgen WILDAU, Berlin (DE); Gerald Czygan, Buckenhof (DE); Michael Diebold, Berlin (DE); Michael Lippert, Ansbach (DE); Claus Harder, Uttenreuth (DE); Thomas Schmitz-Rode, Aachen (DE)

Correspondence Address: DALINA LAW GROUP, P.C., 7910 IVANHOE AVE. #325, LA JOLLA, CA, 92037, US

(21) Appl. No.: 12/190,113

(22) Filed: Aug. 12, 2008

(30) Foreign Application Priority Data
Aug. 17, 2007 (DE) 10 2007 038 801.4

(51) Int. Cl. A61B 5/0215

(52) U.S. Cl. 600/486

(57) ABSTRACT

Implantable pressure measuring unit for internal pressure measurement in a blood vessel or heart of a patient, having a pressure sensor having electrical signal output, fixing means adapted to the intended measurement location for fixing the pressure sensor, a power supply unit of the pressure sensor, a signal detection unit connected by a line to the signal output of the pressure sensor, and a transmitting unit connected to a measurement data output of the signal detection unit, in particular for wireless transmission of measurement data to an analysis unit, in particular outward from the patient body.

This application takes priority from German Patent Application DE 10 2007 038 801.4, filed 17 Aug. 2007, the specification of which is hereby incorporated herein by reference.

### BACKGROUND OF THE INVENTION

1. Field of the Invention

The invention relates to an implantable pressure measuring unit for internal pressure measurement in a blood vessel or in the heart and, furthermore, a configuration expanded with an analysis unit (a “patient device”).

2. Description of the Related Art

The continuous monitoring of the physical status of patients having chronic illnesses is essential for ensuring an optimum therapy. Especially patients having heart diseases require monitoring of the cardiac and circulatory status to check and control their medication therapy, be able to predict acute worsening, and prevent decompensation. For patients having cardiac damage, diverse monitoring concepts have been suggested, inter alia, the periodic detection of the physical status in the hospital, but also monitoring using external measuring or monitoring units, for example, for checking the weight, the blood pressure, the time dependence of the pulse, etc. In this context, “telemedical” concepts for the remote monitoring of the patient in his normal living environment have also been discussed for some time.

In many cases, implantable measuring units are preferred over other methods, because they determine the relevant variables directly in the body and therefore may fundamentally operate more precisely and reliably than external methods. In addition, these methods may largely be automated in the current prior art and run with data remote monitoring, without the patient himself or the medical personnel monitoring him having to significantly intervene.

For the monitoring of the hemodynamic status of patients having heart diseases, the blood pressure represents one of the most important variables which is to be monitored. Ideally, the end-diastolic pressure in the left ventricle (LV) or the pressure in the left atrium (LA) is to be detected. Because the access to the left side of the heart is subject to significant risk, monitoring the blood pressure in the pulmonary artery (PA) has been established as sufficiently reliable means for monitoring the delivery rate of the left heart. The PA pressure correlates sufficiently well with the LA pressure under the assumption that the lung status is stable.

Pressure sensors for detecting the internal pressure in a liquid vessel are known in a great manifold and are also in practical use in technology. Many of the solutions typical in technology are not transferable without further measures to measuring tasks in the living organism, however, because they are not compact enough, consume too much energy in operation, or are too susceptible to impairments of their function by biological material or display other shortcomings in this special field of use.

Pressure measuring units for use in the living organism, especially also in human blood vessels, have also been known for some years. In this context, reference is made to recent patent publications EP 1117982 A1, US 2002/0045921, U.S. Pat. No. 6,645,143, or U.S. Pat. No. 6,743,180, solely for exemplary purposes.

### BRIEF SUMMARY OF THE INVENTION

The object of the invention is to provide an improved pressure measuring unit having the above-mentioned function and a corresponding measuring configuration, which in particular operate precisely and reliably over a long period of time, are cost-effective to produce, and may be used easily and safely in the body of a patient.

This object is achieved by a pressure measuring unit having the features of Claim 1 and a configuration having the features of Claim 22. Expedient refinements of the idea of the invention are the subject matter of the dependent claims.

The invention includes the essential idea, for achieving the object of internal pressure measurement (in vivo) in a blood vessel or possibly also the heart, providing a pressure sensor having electrical signal output and associated power supply unit, whose signal is detected in the body and then transmitted wirelessly outward from the body for further analysis/use. With a sufficiently broad understanding of the invention, nothing is to say that the measurement data may not also be used inside the body—for example, to control a physical function support unit—and no transmission outward from the body has to be performed in this design.

The use of a capacitive pressure sensor is currently viewed as expedient, fundamentally, however, pressure sensors functioning according to other physical action principles, in particular managing without moving parts, also come into consideration, such as piezoelectric or piezoresistive sensors.

An essential aspect of the invention is providing fixing means for fixing the pressure sensor which are adapted to the intended measurement location. An expandable vascular wall implant (stent) connected to a pressure sensor body comes into consideration as such fixing means. Stents of this type have been known for some time in numerous embodiments and are on the market, so that commercially available fixing means having suitable construction and fitting geometry may be used without further measures. The fundamental usage location—such as the pulmonary artery—plays a decisive role in the selection, however, the individual anatomy of the individual patient is also to be considered.

This also applies for further embodiments of the fixing means, of course, for example, as elastically protruding or expandable vascular wall supports, for example, in the form of a “tripod” made of silicone. This applies in the same way for an implementation of the fixing means as a support spiral or helix or the like, which are distinguished by elastically deformable curved sections, which allow implantation without problems and are to press against the corresponding vascular wall in the inserted state of the pressure measuring unit.

Fixing means in the broader meaning which may produce the positioning of the pressure sensor in a suitable positional relationship to the wall of a blood vessel (or heart) without wall contact, for example, a flow-favorable embodiment of the pressure sensor body or flow conduction means thereon which cause its dynamic positioning in a bloodstream, are also noteworthy. Moreover, balloon-type fixing means also come into consideration, which link the effect of a dynamic flow-guided fixing with that of fixing on the wall.

In order, especially using fixing means acting on the wall, to be able on one hand to position the pressure sensor during its use in a suitable position and stably in the blood vessel and on the other hand to be able to remove it again easily after ending the measuring task, a removable connection is preferably provided between the pressure sensor body and the fixing means. This ensures that the pressure sensor may be explanted without injuring the vascular wall (on which the fixing means then remain). Alternatively, the pressure sensor may also be connected permanently to the fixing means in such a way that it may be explanted together with the fixing means with little injury, in that the fixing means may be detached easily from the wall, i.e., from the tissue. The fixing means may be implemented as retractable or unscrewable active fixing means or alternatively formed as passive fixing means projecting from the pressure sensor body which may be folded up.

In a further design of the invention, the signal detection unit has a separate housing which is implemented for implantation remotely from the pressure sensor, outside the blood vessel or heart. The separate housing may be situated in the rib cage of the patient, for example. In a further design, in addition to the signal detection unit, it receives the power supply unit of the pressure sensor, and a power supply line connection is provided between the power supply unit and the pressure sensor. More preferably, a control unit for controlling the sensor and transmitting functions and optionally the power supply is additionally provided in the housing of the signal detection unit. Finally, it is especially advisable if the housing is the housing of an implantable therapy device, in particular a cardiac stimulation device. Especially if the pressure measuring unit is required in connection with a function of such a therapy device, the production and implantation outlay is thus decreased significantly.

In an alternative embodiment thereto, the power supply unit, signal detection unit, transmitting unit, and a control unit for controlling the sensor and telemetry functions, and optionally the power supply are accommodated in a pressure sensor body. It is obvious that special attention is then to be paid to miniaturizing the listed functional units, to be able to design the pressure measuring unit integrated in this meaning as compact overall.

In a further embodiment of the invention, the signal detection unit has a pre-processing unit and a measured value buffer memory for measured value preprocessing and internal buffering connected downstream. This may be advisable both in the integrated embodiment of the measuring unit and also in the embodiment in which the actual pressure sensor and the remaining components do not have an integrated construction. It contributes especially to reducing the data transmission effort between the body of the patient and an external analysis unit, because the data collection and preprocessing, in particular a mean value calculation over specific measurement periods, may be performed intracorporeally.

In a further design of the invention, the pressure sensor and optionally the fixing means have a coating which inhibits endothelialization and/or thrombosis. Although the suggested measuring unit is distinguished by a comparatively high insensitivity in relation to deposits of biological material, if a pressure sensor without moving parts is used, these accumulations are finally capable of impairing the function even in the suggested unit if they exceed a specific amount. Therefore, a suitable coating or encapsulation of the entire pressure sensor body or in any case the sections which record the measuring signal to prevent deposits or to prevent the thrombosis is valuable for long-term operation at high measuring precision and reliability and/or to prevent thrombi from detaching.

From the aspect of an efficient, cost-effective power supply of the pressure measuring unit, it is preferable for the power supply unit to have a primary or secondary element. In regard to the wide distribution of lithium ion elements in pacemakers or other implantable therapy devices, the selection of such a power supply also appears expedient for the suggested pressure measuring unit. In combination therewith, but also independently therefrom, the power supply may have a power receiving unit for wireless power supply from outside the body, in particular for the wireless charging of a secondary element. This may be an inductive unit, for example, as is typical in body care devices and is commercially available cost-effectively.

In a further design, the pressure measuring unit comprises at least one second sensor for measuring an additional variable in the body of the patient. A further pressure sensor may be provided as the second sensor in a Prandtl tube configuration with the first pressure sensor for obtaining a flow velocity signal. Alternatively or also in combination therewith, a temperature sensor may also be provided for determining the blood temperature and/or for temperature compensation of the pressure measurement signal. By providing additional sensors, on one hand a contribution may be provided to expanding the measured variable spectrum and on the other hand a contribution may be provided to increasing the informative power of the results of the pressure measurement. The possibilities of such a multi-sensor unit are in no way exhausted by the exemplary variants cited here.

The overall configuration suggested in the scope of the invention comprises, in addition to the pressure measuring unit, an analysis unit implemented for extracorporeal placement, which has an external telemetry unit for measurement data and optionally also control signal connection to the transmitting unit of the pressure measuring unit. It preferably contains an atmospheric pressure sensor and a pressure compensation unit for calculating an internal pressure value corrected for atmospheric pressure. Additionally or alternatively thereto, a temperature compensation unit connected to the pressure sensor and a temperature sensor of the implanted pressure measuring unit may be provided for calculating an internal pressure value compensated for blood temperature.

In addition to these designs, which have the goal of providing a result of the pressure measurement having more informative power, the analysis unit may also have a secondary variable analysis unit connected to the pressure sensor and/or further sensors of the implantable pressure measuring unit. Corresponding secondary variables may be of significant value for deriving therapeutic measures from the results of the internal pressure measurement. In this meaning, for example, the secondary variable analysis unit may be implemented to determine the beat volume and/or the ejection power of the right ventricle by pulse shape analysis of the pressure measurement signal of the pressure measuring unit.

### BRIEF DESCRIPTION OF THE DRAWINGS

Advantages and special features of the invention also result from the following—partially only schematic—description of preferred exemplary embodiments on the basis of the figures. In the figures:

FIG. 1 shows a schematic illustration of an embodiment of a measuring configuration according to the invention,

FIG. 2 shows a further illustration of the measuring configuration in its configuration in/on the body of a patient,

FIG. 2A shows a detail illustration of the positioning of the pressure sensor in a blood vessel of the patient, and

FIG. 3 shows a schematic sketch of a two-sensor measuring head.

### DETAILED DESCRIPTION OF THE INVENTION

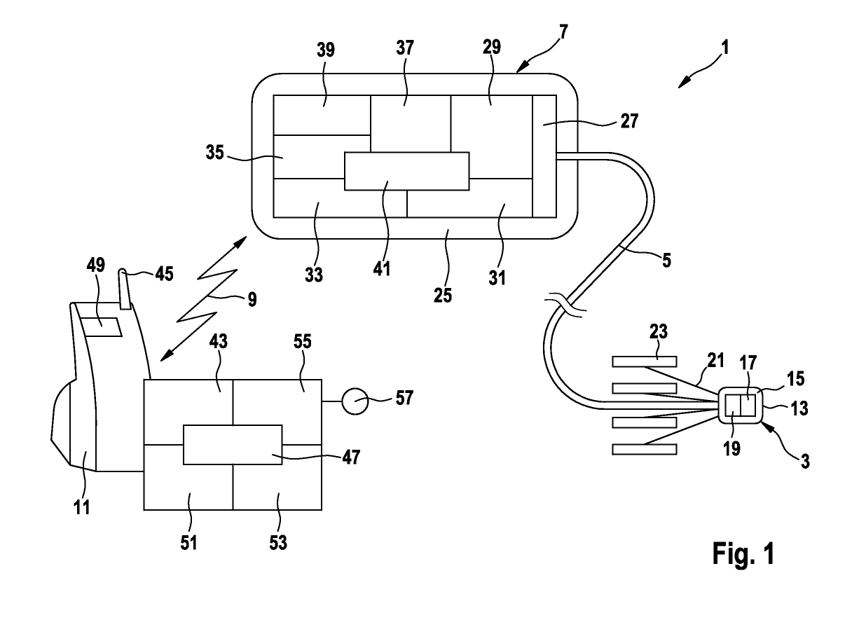

FIG. 1 shows an outline of a measuring configuration 1 for internal pressure measuring in a blood vessel of a patient, whose main components are a pressure measuring head 3, an implantable unit 7 connected thereto via a line 5, and an extracorporeal analysis unit (patient device) 11 connected thereto via a telemetry link 9.

The measuring head 3 comprises a pressure sensor body 13 having a biocompatible and thrombosis-inhibiting encapsulation 15, in which a capacitive pressure sensor 17 and an interface unit 19 are received. The pressure sensor body 13 is removably connected via connection means 21 to a stent 23 adapted to the internal dimensions of the pulmonary artery of a patient (which is shown in the already expanded state here).

Like the pressure sensor body 13, the implantable unit 7 has a biocompatible envelope 25 and contains a line connector 27, a high-performance battery 29 as the power supply unit of the pressure sensor 17, a signal detection unit 31, a signal preprocessing unit 33, a buffer memory 35, an HF transceiver unit 37 as the implantable telemetry unit of the telemetry link 9, and an associated antenna 39. Finally, a controller 41 is provided for controlling the functions of the implantable unit 7.

The patient device 11 contains (inter alia) an external HF transceiver unit 43 having associated antenna 45, a controller 47, and an operating and display panel 49. Furthermore it comprises a separate power supply 51 and finally a pressure calculation stage 53, to which a compensation processing stage 55 is assigned, which processes the signal of a connected atmospheric pressure sensor 57 for air pressure compensation of the vascular internal pressure measurement signal, but may also execute further compensation processing procedures (for example, for T compensation on the basis of the signal of an additional T sensor provided in the measuring head).

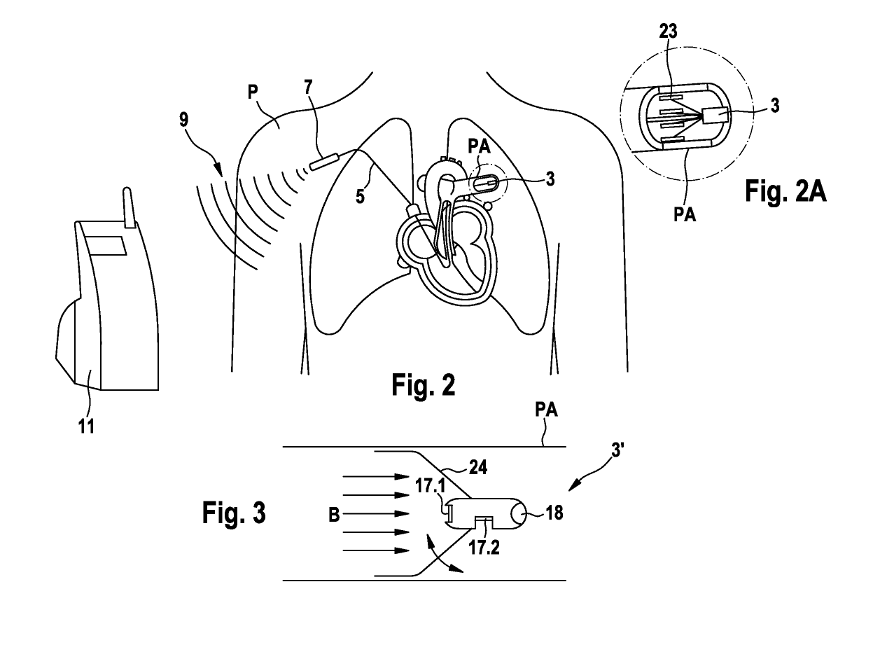

FIGS. 2 and 2A show the positioning of the components of the measuring configuration 1 in relation to the body of a patient P. In addition to the extracorporeal placement of the patient device 11, the placement of the implantable unit 7 in the rib cage, below the clavicle, and the placement of the measuring head 3 in the pulmonary artery PA may be seen. FIG. 2A also shows the support of the measuring head 3 in the center of the pulmonary artery using the stent 23.

FIG. 3 shows a modified embodiment 3′ of the measuring head in the state inserted in the pulmonary artery. The arrows identify the direction of a bloodstream B here. The measuring head 3′ is distinguished by the presence of two pressure sensors 17.1, 17.2 in a spatial configuration like a Prandtl static tube for the combined measurement of the pressure and the flow velocity of the bloodstream B.

Furthermore, the measuring head 3′ contains a T sensor 18 for the simultaneous detection of the blood temperature, whose signal may be used for ascertaining a more precise internal pressure value, namely a T-compensated value; cf. the above description of the measuring configuration 1 in regard to the patient device 11. Finally, a modified embodiment of the fixing means for positioning the measuring head 3′ in the center of the pulmonary artery PA is shown in this figure, which comprises a plurality of vascular wall supports 24 which may be elastically pressed and/or spread. These may be manufactured from a silicone, for example, and possibly shaped onto a silicone envelope (not shown here) of the measuring head 3′.

The embodiment of the invention is not restricted to the examples explained and aspects emphasized here, but rather is also possible in a plurality of alterations which are in the scope of typical measures of one skilled in the art.

What is claimed is

1. An implantable pressure measuring unit (3, 7; 3′) for measurement of internal pressure in a blood vessel or heart of a patient (P), comprising:
a pressure sensor (17; 17.1, 17.2) having an electrical signal output;
a fixing means (23; 24) adapted to fix the pressure sensor at an intended measurement location;
a power supply unit (29) of the pressure sensor;
a signal detection unit (31) connected to the electrical signal output of the pressure sensor by a line; and,
a transmitting unit (37) connected to a measurement data output of the signal detection unit, wherein the transmitting unit is configured for wireless transmission of measurement data to an analysis unit (11) outside of a patient's body.

2. The implantable pressure measuring unit according to claim 1, wherein the fixing means comprises a flow guiding means situated on a pressure sensor body wherein
the flow guiding means is configured to bloodstream-induced dynamically center the pressure sensor in a blood vessel or heart, or,
the pressure sensor is configured to favorable flow bloodstream-induced center the pressure sensor in a blood vessel or heart.

3. The implantable pressure measuring unit according to claim 1, wherein the fixing means have
vascular wall supports (24) that elastically project or are spreadable from a pressure sensor body (13),
a support spiral or helix, or,
a vascular wall anchor for introduction into a wall of a blood vessel or heart, on a branch section.

4. The implantable pressure measuring unit according to claim 1, wherein the fixing means comprises a stent (23) connected to a pressure sensor body (13).

5. The implantable pressure measuring unit according to claim 1, wherein the fixing means (23, 24) are dimensioned to fix the pressure sensor in a pulmonary artery (PA).

6. The implantable pressure measuring unit according to claim 1, wherein the pressure sensor (17) is removably (21) connected to the fixing means (23) in such a way that the pressure sensor may be pulled out of the blood vessel while the fixing means remain therein.

7. The implantable pressure measuring unit according to claim 1, wherein the pressure sensor (17) is permanently connected to the fixing means (23) in such a way that it may be explanted together with the fixing means with little injury, in that the fixing means may be detached easily from a wall, or from tissue.

8. The implantable pressure measuring unit according to claim 7, wherein the fixing means (23) that are permanently connected to the pressure sensor are implemented as active, retractable, or unscrewable fixing means.

9. The implantable pressure measuring unit according to claim 7, wherein the fixing means (23) that are permanently connected to the pressure sensor are shaped as passive fixing means that project from a pressure sensor body, wherein the fixing means may be folded in.

10. The implantable pressure measuring unit according to claim 1, wherein the signal detection unit (31) has a separate housing (7) which is implemented for implantation remotely from the pressure sensor, outside a blood vessel or heart.

11. The implantable pressure measuring unit according to claim 10, wherein the separate housing (7) of the signal detection unit (31) receives the power supply unit (29) of the pressure sensor (17; 17.1, 17.2) and a power supply line connection (5) is provided between the power supply unit and the pressure sensor.

12. The implantable pressure measuring unit according to claim 10, further comprising:
a control unit (41) configured to control the pressure sensor and transmitting unit; and,
optionally, wherein the power supply is provided in the separate housing (7) of the signal detection unit (3 1).

13. The implantable pressure measuring unit according to claim 10, wherein the separate housing is the housing of an implantable therapy device, or a cardiac stimulation device.

14. The implantable pressure measuring unit according to claim 1, further comprising:
a control unit configured to control the pressure sensor and transmitting unit and wherein the power supply unit, signal detection unit, transmitting unit, and the control unit are received in a pressure sensor body; and,
optionally, wherein the power supply is received in the pressure sensor body.

15. The implantable pressure measuring unit according to claim 1, further comprising:
a preprocessing unit (33) configured to preprocess a measured value;
a measured value buffer memory (35) configured to internally buffering the measured value; and,
wherein the preprocessing unit and measured value buffer memory are connected downstream from the signal detection unit (3 1).

16. The implantable pressure measuring unit according to claim 1, wherein:
the pressure sensor (17; 17.1, 17.2) comprises a coating which inhibits endothelialization; and,
optionally, the fixing means (23, 24) comprise a coating which inhibits endothelialization.

17. The implantable pressure measuring unit according to claim 1, wherein the power supply unit (29) has a primary or secondary element.

18. The implantable pressure measuring unit according to claim 1, wherein the power supply unit comprises an energy receiving unit for wireless energy supply from outside the patient's body, in particular for wireless charging of a secondary element in the power supply.

19. The implantable pressure measuring unit according to claim 1, further comprising a second sensor (17.2) configured to measure an additional variable in the patient's body.

20. The implantable pressure measuring unit according to claim 19, wherein the second sensor comprises a further pressure sensor (17.2) in a Prandtl tube configuration with the pressure sensor (17.1) wherein the pressure sensor and further pressure sensor are configured to obtain a flow velocity signal.

21. The implantable pressure measuring unit according to claim 19, further comprising a temperature sensor (18) configured to determine a temperature of blood and/or for temperature compensation of a pressure measurement signal.

22. A configuration (1) for measurement of internal pressure in a blood vessel (PA) or heart of a patient (P), having an implantable pressure measuring unit (3, 7; 3′) according to claim 1 further comprising:
an analysis unit (11) implemented for extracorporeal placement, which has an external telemetry unit (43) for measurement data connection; and,
optionally, a control signal connection from the analysis unit to the transmitting unit (37) of the pressure measuring unit.

23. The configuration according to claim 22, wherein the analysis unit (11) has an atmospheric pressure sensor (57) and a pressure compensation unit (55) wherein the analysis unit is configured to calculate an internal pressure value corrected for atmospheric pressure.

24. The configuration according to claim 22, wherein the analysis unit (11) has a temperature compensation unit (55), connected to the pressure sensor (17; 17.1, 17.2) and a temperature sensor (18) of the implantable pressure measuring unit (3, 7; 3′), wherein the analysis unit is configured to calculate an internal pressure value compensated for blood temperature.

25. The configuration according to claim 22, wherein the analysis unit (11) has a secondary variable analysis unit connected to the pressure sensor (17; 17. 1) and/or further sensors (17.2; 18) of the implantable pressure measuring unit.

26. The configuration according to claim 25, wherein the secondary variable analysis unit is implemented to determine a beat volume and/or an ejection power of a right ventricle by pulse shape analysis of a pressure measurement signal of the implantable pressure measuring unit.
